(19) United States
(12) Patent Application Publication
(10) Pub. No.: US 2011/0155924 A1
(43) Pub. Date: Jun. 30, 2011

(54) STERILIZING APPARATUS FOR STERILIZING KITCHEN UTENSILS

(76) Inventor: Tien-Jen Lo, Kaohsiung City (TW)

(21) Appl. No.: 12/750,428

(22) Filed: Mar. 30, 2010

(30) Foreign Application Priority Data
Dec. 31, 2009 (TW) 098224986

(51) Int. Cl. A61L 2/10

(52) U.S. Cl. 250/455.11

(57) ABSTRACT

A sterilizing apparatus is adapted for sterilizing kitchen utensils, and includes a hollow housing cooperating with a bottom seat and a top cover to define an accommodating space for receiving the kitchen utensils, and a transparent plate impermeable to ultraviolet light and disposed in front of and covering openably the accommodating space. Two sterilizing lamp mechanisms are disposed in the accommodating space, and are spaced apart from each other in a left-to-right direction, such that the kitchen utensils are disposed between the sterilizing lamp mechanisms. The sterilizing lamp mechanisms emit ultraviolet rays onto the kitchen utensils.

### CROSS-REFERENCE TO RELATED APPLICATION

This application claims priority of Taiwanese Application No. 098224986, filed on Dec. 31, 2009.

### BACKGROUND OF THE INVENTION

1. Field of the Invention

This invention relates to a sterilizing apparatus, and more particularly to a sterilizing apparatus for sterilizing kitchen utensils.

2. Description of the Related Art

A commercially available utensil holder is used to retain kitchen utensils (e.g., chopping boards, knives, spoons, glasses, and cups) thereon. Such a utensil holder can be provided with an ultraviolet lamp for sterilizing the kitchen utensils.

Referring to FIG. 1, a conventional utensil holder 1 is configured as a knife block, and includes a block body 11 having a top surface formed with a plurality of knife-receiving grooves 110 allowing for insertion of a plurality of knives, respectively, and a bottom seat 12 connected fixedly to a bottom end of the block body 11 by a plurality of lock bolts 13. An ultraviolet lamp (not shown) is disposed within the bottom seat 12 for generating and emitting ultraviolet rays onto the knives for sterilizing purposes. When cleaning of the interior of the block body 11 or replacement of the ultraviolet lamp is desired, it is necessary to remove the bolts 13, thereby resulting in a troublesome disassembly process. Furthermore, since the widths of the grooves 110 are relatively narrow, and since specific tools are required to clean the grooves 110, the interior of the block body 11 is not convenient to clean.

### SUMMARY OF THE INVENTION

The object of this invention is to provide a sterilizing apparatus for sterilizing kitchen utensils that is convenient to use.

According to this invention, a sterilizing apparatus is adapted for sterilizing kitchen utensils, and includes a hollow housing cooperating with a bottom seat and a top cover to define an accommodating space for receiving the kitchen utensils, and a transparent plate impermeable to ultraviolet light and disposed in front of and covering openably the accommodating space. Two sterilizing lamp mechanisms are disposed in the accommodating space, and are spaced apart from each other in a left-to-right direction, such that the kitchen utensils are disposed between the sterilizing lamp mechanisms. The sterilizing lamp mechanisms emit ultraviolet rays onto the kitchen utensils.

Since the transparent plate is openable to allow for easy access into the accommodating space, the accommodating space is convenient to clean, and the sterilizing lamp mechanisms are convenient to replace.

### BRIEF DESCRIPTION OF THE DRAWINGS

These and other features and advantages of this invention will become apparent in the following detailed description of the preferred embodiments of this invention, with reference to the accompanying drawings, in which:

FIG. 1 is a perspective view of a conventional utensil holder that is configured as a knife block;

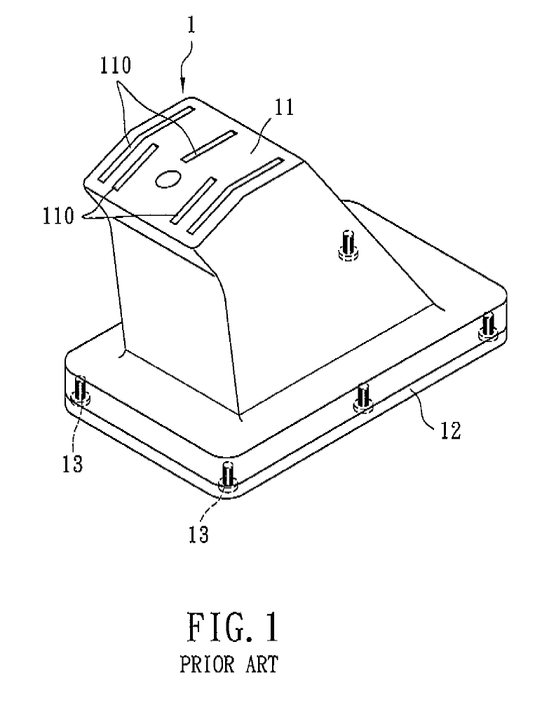

FIG. 2 is a perspective view of the first preferred embodiment of a sterilizing apparatus for sterilizing kitchen utensils according to this invention;

FIG. 3 is a front view of the first preferred embodiment, illustrating how a plurality of knives are confined within utensil-receiving holes in a positioning cover;

FIG. 4 is a partly exploded perspective view of the first preferred embodiment;

FIG. 5 is a fragmentary, partly exploded perspective view of the first preferred embodiment;

FIG. 6 is a fragmentary sectional view of the first preferred embodiment;

FIG. 7 is a fragmentary bottom perspective view of the first preferred embodiment;

FIG. 8 is a perspective view of the second preferred embodiment of a sterilizing apparatus for sterilizing kitchen utensils according to this invention;

FIG. 9 is a front view of the third preferred embodiment of a sterilizing apparatus for sterilizing kitchen utensils according to this invention; and

FIG. 10 is a top view of the third preferred embodiment, illustrating how a transparent plate is opened.

### DETAILED DESCRIPTION OF THE PREFERRED EMBODIMENTS

Before the present invention is described in greater detail in connection with the preferred embodiments, it should be noted that similar elements and structures are designated by like reference numerals throughout the entire disclosure.

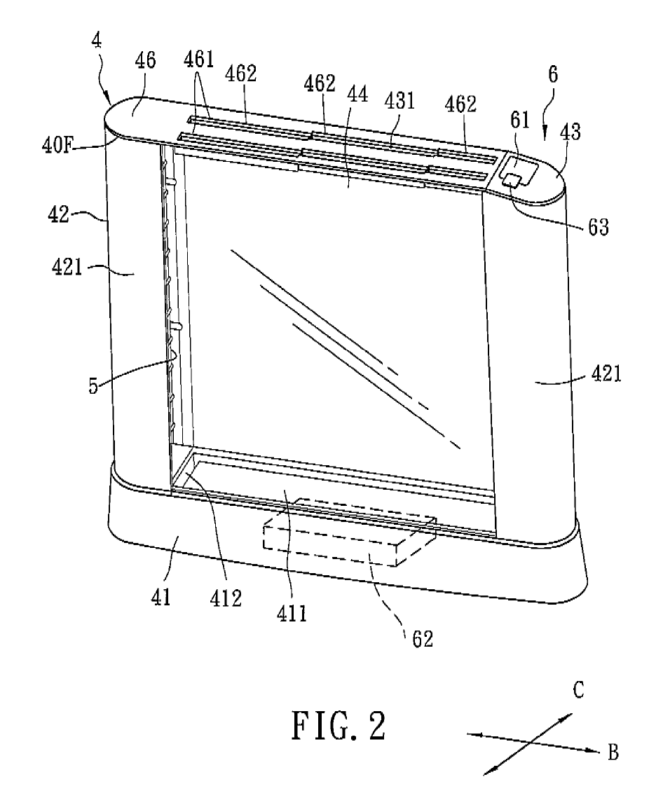

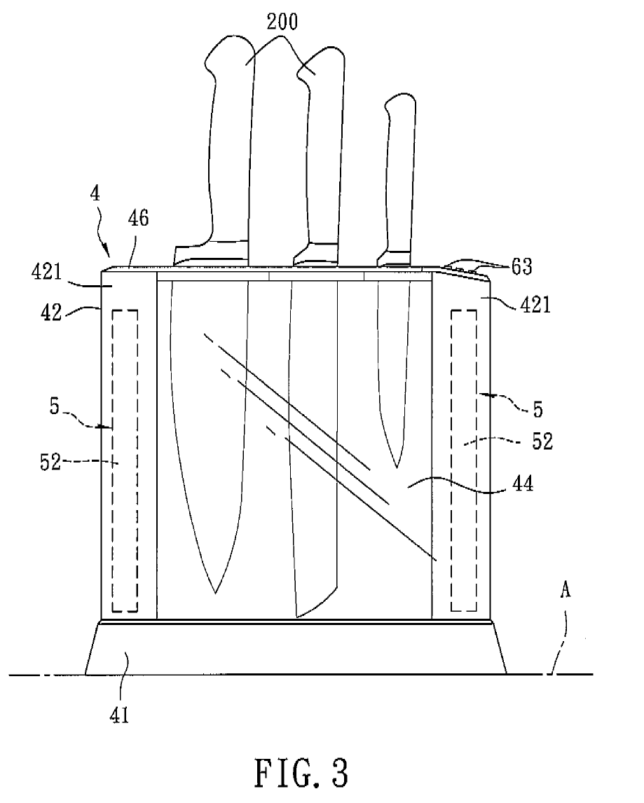

Referring to FIGS. 2 and 3, the first preferred embodiment of a sterilizing apparatus according to this invention is used to hold a plurality of knives 200.

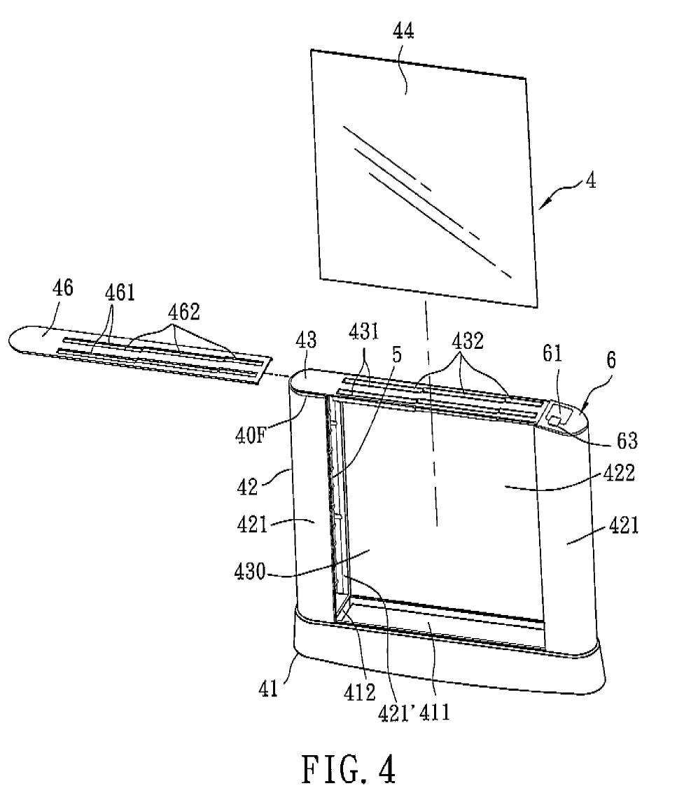

With additional reference to FIGS. 4, 5, and 6, the sterilizing apparatus includes a housing mechanism 4, two sterilizing lamp mechanisms 5, and a control mechanism 6 disposed on the housing mechanism 4 and connected electrically to the sterilizing lamp mechanisms 5.

The housing mechanism 4 includes an elongated bottom seat 41 extending in a left-to-right direction (B) and having a bottom surface abutting against a support surface (A), a hollow housing 42 disposed on a top end (i.e., top surface) of the bottom seat 41, a top cover 43 disposed fixedly on a top end of the hollow housing 42, a transparent plate 44 disposed removably on the hollow housing 42 and between the bottom seat 41 and the top cover 43, and a positioning cover 46 disposed removably on the top cover 43 so as to prevent movement of the transparent plate 44 relative to the hollow housing 42.

The top surface of the bottom seat 41 has an elongated water-receiving groove 411 extending in the left-to-right direction (B), and a water discharge hole 412 in spatial communication with the water-receiving groove 411 and allowing water to leak out of the bottom seat 41 therethrough.

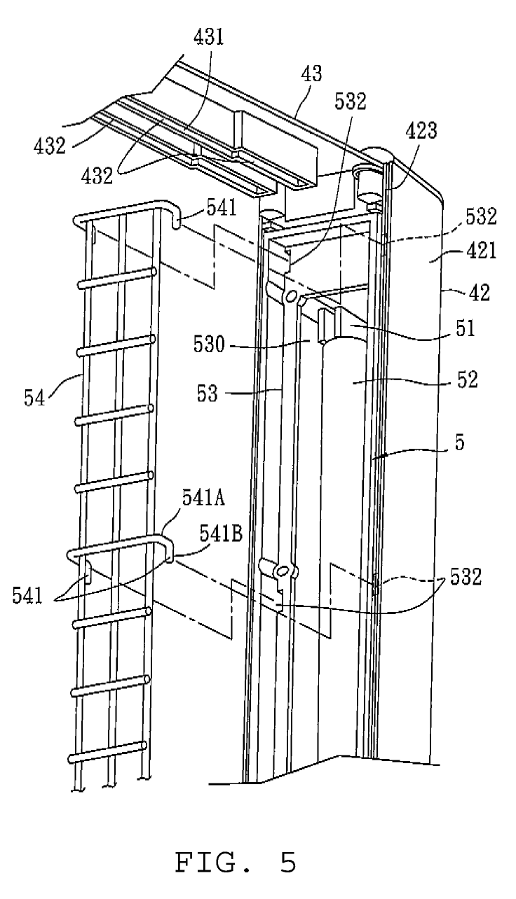

The hollow housing 42 includes two lateral walls 421 spaced apart from each other in the left-to-right direction (B) and disposed fixedly on the top surface of the bottom seat 41, and an upright rear wall 422 interconnecting rear sides of the lateral walls 421. The lateral walls 421 are disposed respectively at left and right sides of the water-receiving groove 411, and are U-shaped in cross-section to define a gap 421′ (see FIG. 4). The gaps 421′ in the lateral walls 421 face toward each other to permit the sterilizing lamp mechanisms 5 to be received respectively therein. Front ends of the lateral walls 421 are formed respectively with two aligned vertical slots 423 (only one is shown in FIG. 5) that permit two opposite sides of the transparent plate 44 to be received respectively and movably therein and that have open upper ends to allow for upward removal of the transparent plate 44 from the hollow housing 42 therethrough.

The top cover 43 cooperates with the hollow housing 42 and the bottom seat 41 to define an accommodating space 430 that is open forwardly. As such, the transparent plate 44 is disposed in front of the accommodating space 430 for covering the same, and the sterilizing lamp mechanisms 5 are disposed respectively in left and right sides of the accommodating spaces 430. Two utensil-receiving holes 431 are formed through the top cover 43. Each of the utensil-receiving holes 431 has a plurality of interconnected longitudinal hole sections 432. Any two adjacent ones of the longitudinal hole sections 432 are misaligned partially from each other in the left-to-right direction (B).

The transparent plate 44 is made of a material that is impermeable to ultraviolet light. Since the plate 44 is transparent, the knives 200 and operation of the sterilizing lamp mechanisms 5 are visible through the transparent plate 44. In this embodiment, the material is glass.

An assembly of the hollow housing 42 and the cover plate 43 is formed with horizontal front and rear slide slot units (40F, 40R) disposed respectively and immediately under front and rear sides of the cover plate 43.

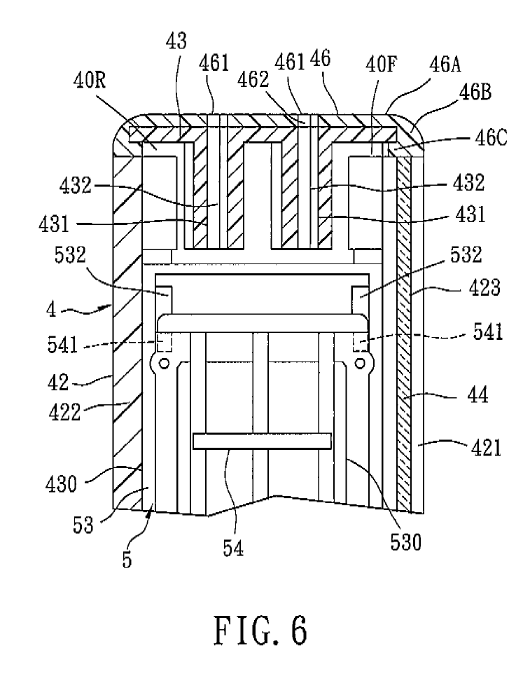

With particular reference to FIGS. 2, 4, and 6, the positioning cover 46 has a top wall (46A) abutting against a top surface of the top cover 42, two sidewalls (46B) extending respectively and downwardly from front and rear sides of the top wall (46A) and flanking the top cover 43, and a pair of front and rear slide plate portions (46C) extending respectively from lower ends of the sidewalls (96A) toward each other and press-fitted respectively into the front and rear slide slot units (40F, 40R), respectively. The front slide plate portions (46C) abut against a top end of the transparent plate 44 for preventing upward removal of the transparent plate 44 from the hollow housing 42. The top wall (46A) has two utensil-receiving holes 461 identical in shape to and aligned respectively with the utensil-receiving holes 431 in the top cover 43. That is, each of the utensil-receiving holes 461 has a plurality of longitudinal hole sections 462. It should be noted that, the positioning cover 46 can be easily assembled to and disassembled from the top cover 43 without any tools, thereby resulting in convenience during use thereof. Since a front end of the cover plate 43 is disposed behind the transparent plate 44, as shown in FIG. 6, when the positioning cover 46 is dissembled from the top cover 43, the transparent plate 44 can be removed upwardly from the hollowing housing 42 in a vertical direction.

The sterilizing lamp mechanisms 5 are used to generate and emit ultraviolet rays onto the knives 200. Since the knives 200 are disposed between the sterilizing lamp mechanisms 5, ultraviolet radiation of relatively uniform intensity can be provided on the knives 200.

Each of the sterilizing lamp mechanisms 5 includes two electrode units 51 connected respectively and fixedly to the lateral walls 421 and arranged one above the other, a lamp tube 52 connected removably and electrically between the electrode units 51 and driven by the control mechanism 6 to generate ultraviolet rays, a loop-shaped mounting seat 53 spaced apart from and adjacent to the lamp tube 52 and located between the lamp tube 52 and the other one of the sterilizing lamp mechanisms 5, and a protective net 54 disposed removably on a side of the mounting seat 53 distal from the lamp tube 52.

The mounting seat 53 has a central opening 530 that is sized to allow for extension of the lamp tube 52 therethrough and that is covered by the protective net 54. The mounting seat 53 has two vertical rows of retaining holes 532 formed therethrough. The protective net 54 has two vertical rows of retaining hooks 541 engaging respectively the retaining holes 532 in the mounting seat 53 so as to prevent contact of the knives 200 with the lamp tube 52. With particular reference to FIG. 5, in this embodiment, each of the positioning hooks 541 is L-shaped, and has a horizontal hook portion (541A) extending through the corresponding retaining hole 532, and a vertical hook section (541B) extending downwardly from an end of the horizontal hook section (541A) and having a length smaller than the vertical length of the corresponding retaining hole 532, so as to allow for movement of the vertical hook section (541B) through the corresponding retaining hole 532. As such, the protective net 54 is easy and convenient to be assembled to and disassembled from the mounting seat 53.

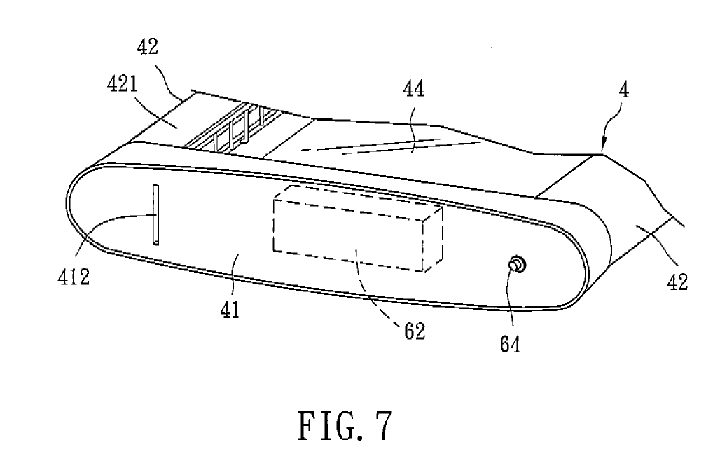

With further reference to FIG. 7, the control mechanism 6 includes a display 61 disposed on an end portion of a top surface of the top cover 43, a controller 62 disposed within the bottom seat 41 and connected electrically to the display 61 and the electrode units 51 of the sterilizing lamp mechanisms 5, a plurality of control keys 63 disposed on the top surface of the top cover 43 and in proximity to the display 61 and connected electrically to the controller 62, and a relay 64 disposed on the bottom seat 41 and projecting downwardly from the bottom surface of the bottom seat 41.

The control keys 63 can be pressed to control the controller 63 for turning on or off the sterilizing lamp mechanisms 5, and setting the turn-on period (e.g., 30 minutes, one hour, and two hours) of the sterilizing lamp mechanisms 5. Due to electrical connection between the display 61 and the controller 62, the display 61 can provide some information, for example, the sterilizing lamp mechanisms 5 are in the ON or OFF state, and the turn-on period of the sterilizing lamp mechanisms 5.

The relay 64 is configured as a switch. When the relay 64 is pressed, e.g., by the support surface (A), it emits a corresponding signal to the controller 62 so that the sterilizing lamp mechanisms 5 are turned on under control of the controller 62. When the housing mechanism 4 is toppled to result in removal of the relay 64 from the support surface (A), the relay 64 emits a corresponding signal to the controller 62 so that the sterilizing lamp mechanisms 5 are turned off under control of the controller 62. Hence, contact between water remaining in the accommodating space 430 and the sterilizing lamp mechanisms 5 can be prevented, thereby promoting safety during use of the sterilizing apparatus.

The knives 200 are inserted into the longitudinal hole sections 432, 462 of the hole-receiving holes 431, 461. Since any two adjacent longitudinal hole sections 432, 462 are misplaced partially from each other, any portion of each of the knives 200 is subjected to emission of the ultraviolet rays from at least one of the sterilizing lamp mechanisms 5 to thereby promote the sterilizing effect significantly.

When it is desired to clean the interior of the housing mechanism 4 or replace either of the lamp tubes 52, the positioning cover 46 is first removed from the top cover 43. Next, the transparent plate 44 is removed upwardly from the hollow housing 42 to allow for the cleaning or replacement operation. As such, no tool is required to open the accommodating space 430. In the case of the replacement operation, the protective net 54 can be removed by moving upwardly and then horizontally away from the corresponding mounting seat 53. Once the protective net is removed from the accommodating space 430, the corresponding lamp tube 52 can be replaced with ease.

Furthermore, since the plate 44 is transparent, the knives 200 are visible therethrough. Hence, the user can take the desired knife 200 quickly out of the housing mechanism 4.

In this embodiment, due to the inclusion of the bottom seat 41, the sterilizing apparatus is placed on a table. Alternatively, the bottom seat 41 may be replaced with a hanging device that is disposed on the rear wall 422, such that the sterilizing apparatus can be hung on a wall surface. If this occurs, the relay 64 is useless, and can be omitted.

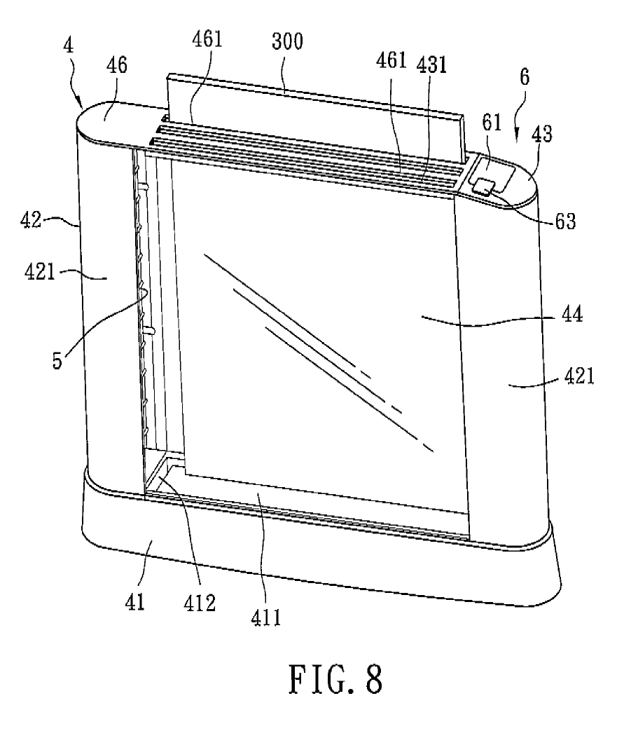

FIG. 8 shows the second preferred embodiment of a sterilizing apparatus for sterilizing kitchen utensils according to this invention, which is different from the first preferred embodiment in the number and shape of the utensil-receiving holes 431, 461 of the top cover 43 and the positioning cover 46.

In this embodiment, each of the top cover 43 and the positioning cover 46 is formed with three aligned straight utensil-receiving holes 431, 461 that permit three chopping board 300 (only one is shown) to be inserted respectively thereinto.

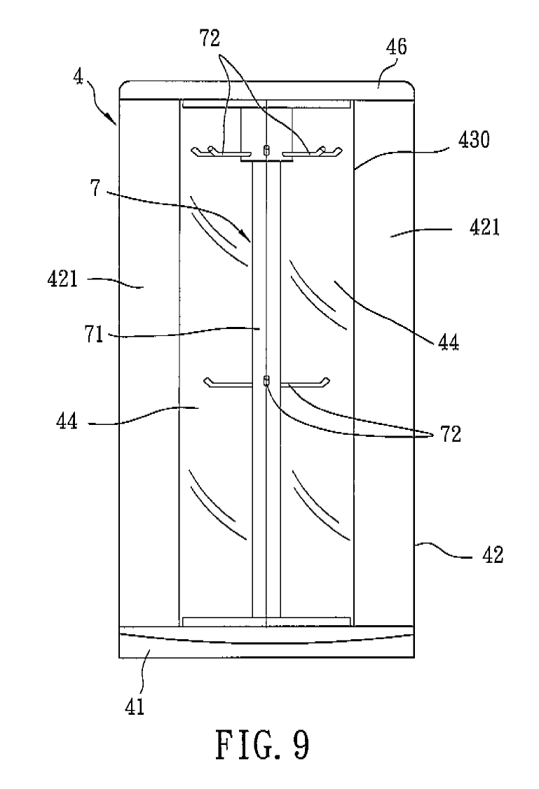

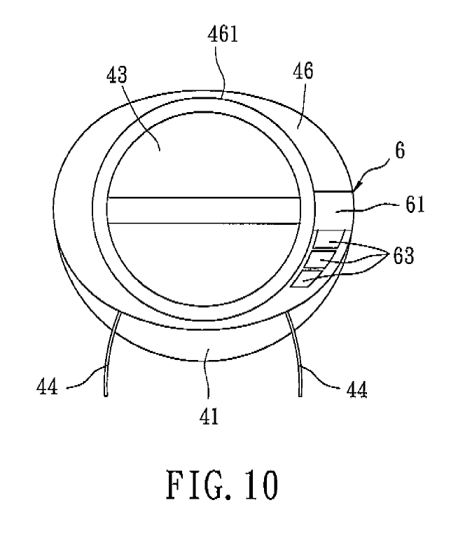

FIGS. 9 and 10 show the third preferred embodiment of a sterilizing apparatus for sterilizing kitchen utensils according to this invention, which is different from the first preferred embodiment in the construction of the housing mechanism 4 and in addition of a hanger mechanism 7.

Unlike the first preferred embodiment, in this embodiment, each of the lateral walls 421 of the housing mechanism 4 is not formed with any vertical slot, and the housing mechanism 4 includes two modified transparent plates 44 connected respectively and pivotally to the front ends of the lateral walls 421 and cooperating with each other to cover openably the accommodating space 430. The transparent plates 44 are pivotable away from each other to allow for access into the accommodating space 430.

The positioning cover 46 is loop-shaped, and has an elliptical profile. In this embodiment, the positioning plate 46 is disposed fixedly on a top end of the hollow housing 42, and cooperates with the hollow housing 42 and the bottom seat 41 to define an accommodating space 430. A circular hole 461 is formed through the center of the positioning plate 46, and is in spatial communication with the accommodating space 430. The top cover 43 is disposed rotatably on the positioning cover 46 for covering the circular hole 461, and is removable upwardly from the positioning cover 46.

The hanger mechanism 7 includes an upright shaft 71 disposed in the accommodating space 430 and having a top end connected fixedly to the top cover 43, and a plurality of hangers 72 connected fixedly to the shaft 71 for hanging the kitchen utensils. As such, the top cover 43 is co-rotatable with the shaft 71 and the hangers 72. The display 61 and the control keys 62 of the control mechanism 6 are disposed on a top surface of the positioning cover 46.

When maintenance and repair of the sterilizing lamp mechanisms (not shown) are desired, the top cover 43 is separated from the positioning cover 46 to remove the hanger mechanism 7 from the accommodating space 430, and the transparent plates 44 are opened (i.e., pivoted away from each other). When it is desired to take any one of the kitchen utensils from the accommodating space 430, it is only necessary to open the transparent plates 44.

In view of the above, the sterilizing apparatus of this invention has the following advantages:
(1) Since the transparent plate 44 and the modified transparent plates 44 can be opened easily without any tools, the interior of the housing mechanism 4 is convenient to clean, and the sterilizing lamp mechanisms 5 are convenient to replace.
(2) The kitchen utensils are held between the sterilizing lamp mechanisms 5. Thus, the ultraviolet radiation of relatively uniform intensity can be provided on the kitchen utensils.
(3) Since the kitchen utensils are positioned in the accommodating space 430 in such a manner that they are misaligned from each other in the left-to-right direction (B), emission of the ultraviolet rays onto any portion of any one of the kitchen utensils can be ensured.
(4) The ultraviolet rays can be blocked by the transparent plate 44 or the modified transparent plates 44 from leak out of the accommodating space 430.
(5) Due to the presence of the protective net 54, contact between the kitchen utensils and the lamp tubes 52 can be prevented.

With this invention thus explained, it is apparent that numerous modifications and variations can be made without departing from the scope and spirit of this invention. It is therefore intended that this invention be limited only as indicated by the appended claims.

I claim:

1. A sterilizing apparatus adapted for sterilizing kitchen utensils, said sterilizing apparatus comprising:
a housing mechanism including
a bottom seat adapted to be disposed on a support surface,
a hollow housing disposed on a top end of said bottom seat,
a top cover disposed on a top end of said hollow housing so as to cooperate with said hollow housing and said bottom seat to define an accommodating space that is open forwardly, said top cover having at least one utensil-receiving hole that is formed therethrough and that is adapted to permit one of the kitchen utensils to be inserted into said accommodating space therethrough,
a transparent plate disposed removably on said hollow housing and in front of said accommodating space for covering openably said accommodating space in such a manner to allow for upward removal of said transparent plate from said hollow housing, said transparent plate being made of a material that is impermeable to ultraviolet light, and
a positioning cover connected removably to said top cover and abutting against a top end of said transparent plate for preventing the upward removal of said transparent plate from said hollow housing, said positioning cover having a utensil-receiving hole that is formed therethrough, that is in spatial communication with and aligned with said utensil-receiving hole in said top cover, and that is adapted to perm it extension of the one of the kitchen utensils therethrough; and
two sterilizing lamp mechanisms disposed respectively within two opposite sides of said accommodating space and spaced apart from each other in a left-to-right direction such that the one of the kitchen utensils is disposed between said sterilizing lamp mechanisms so as to emit ultraviolet rays from said sterilizing lamp mechanisms onto the one of the kitchen utensils.

2. The sterilizing apparatus as claimed in claim 1, wherein each of said top cover and said positioning cover has a plurality of said utensil-receiving holes that are adapted to permit the kitchen utensils to be extend respectively therethrough.

3. The sterilizing apparatus as claimed in claim 1, wherein said utensil-receiving hole in each of said top cover and said positioning cover is stepped, and has a plurality of interconnected longitudinal hole sections each extending in the left-to-right direction, any two adjacent ones of said longitudinal hole sections being misaligned partially from each other in the left-to-right direction.

4. The sterilizing apparatus as claimed in claim 1, wherein said hollow housing includes two upright lateral walls spaced apart from each other in the left-to-right direction and formed respectively with two aligned vertical slots that permit two opposite sides of said transparent plate to be received respectively and movably therein and that have open upper ends to allow for the upward removal of said transparent plate from said hollow housing therethrough.

5. The sterilizing apparatus as claimed in claim 4, wherein each of said lateral walls has a U-shaped cross-section defining a gap, said gaps in said lateral walls facing toward each other and permitting said sterilizing lamp mechanisms to be received respectively therein, each of said sterilizing lamp mechanisms including two electrode units arranged one above the other, a lamp tube connected electrically between said electrode units for generating said ultraviolet rays, a loop-shaped mounting seat spaced apart from and adjacent to said lamp tube and located between said lamp tube and the other one of said sterilizing lamp mechanisms, and a protective net disposed removably on a side of said mounting seat distal from said lamp tube, said loop-shaped mounting, seat of each of said sterilizing lamp mechanisms having a central opening that is sized to allow for extension of said lamp tube of a corresponding one of said sterilizing lamp mechanisms therethrough and that is covered by said protective net of the corresponding one of said sterilizing lamp mechanisms.

6. The sterilizing apparatus as claimed in claim 1, wherein said mounting seat has at least one vertical row of retaining holes formed therethrough, and said protective net has at least one vertical row of positioning hooks engaging respectively said retaining holes in said mounting seat.

7. The sterilizing apparatus as claimed in claim 6, wherein each of said positioning hooks of said protective net is L-shaped, and has a horizontal hook section extending through a corresponding one of said retaining holes in said mounting seat, and a vertical hook section extending downwardly from an end of said horizontal hook section and having a length smaller than a vertical length of the corresponding one of said retaining holes in said mounting seat, so as to allow for movement of said vertical hook section through the corresponding one of said retaining holes in said mounting seat.

8. The sterilizing apparatus as claimed in claim 1, further comprising a control mechanism, said control mechanism including a display disposed on and exposed outwardly of said housing mechanism, a controller disposed in said housing mechanism and connected electrically to said display and said electrode units, and a plurality of control keys disposed on and exposed outwardly of said housing mechanism and connected electrically to said controller.

9. The sterilizing apparatus as claimed in claim 8, wherein said bottom seat has a bottom surface adapted to abut against the support surface, said control mechanism further includes a relay disposed on said bottom seat and projecting downwardly from said bottom surface of said bottom seat, said relay activating said controller to turn off said sterilizing lamp mechanisms when said housing mechanism is toppled to result in removal of said relay from the support surface.

10. A sterilizing apparatus adapted for sterilizing kitchen utensils, said sterilizing apparatus comprising:
a housing mechanism including
a bottom seat,
a hollow housing disposed on a top end of said bottom seat,
a positioning cover disposed fixedly on a top end of said hollow housing and cooperating with said bottom seat and said hollow housing to define an accommodating space, said positioning cover having a hole formed therethrough and in spatial communication with said accommodating space,
a top cover disposed rotatably on said positioning cover and covering said hole in said positioning cover in such a manner to allow for upward removal thereof from said positioning cover, and
at least one transparent plate disposed pivotally on said hollow housing and in front of said accommodating space for covering openably said accommodating space;
a hanger mechanism including an upright shaft disposed in said accommodating space and having a top end connected fixedly to said top cover, and a plurality of hangers connected fixedly to said shaft and adapted for hanging the kitchen utensils, respectively; and
two sterilizing lamp mechanisms disposed respectively within two opposite sides of said accommodating space and spaced apart from each other in a left-to-right direction such that the kitchen utensils are disposed between said sterilizing lamp mechanisms so as to emit ultraviolet rays from said sterilizing lamp mechanisms onto the kitchen utensils.
